(19) United States
(12) Patent Application Publication
(10) Pub. No.: US 2006/0004529 A1
(43) Pub. Date: Jan. 5, 2006

(54) Method, computer program product with program code segments and computer program product for analysis of a regulatory genetic network of a cell

(76) Inventors: Mathaeus DeJori, Munich (DE); Martin Stetter, Munich (DE)

Correspondence Address: HARNESS, DICKEY & PIERCE, P.L.C., P.O.BOX 8910, RESTON, VA, 20195, US

(21) Appl. No.: 11/155,554

(22) Filed: Jun. 20, 2005

(30) Foreign Application Priority Data
Jun. 23, 2004 (DE) 10 2004 030 296.0

(51) Int. Cl. G06F 19/00; G01N 33/48

(52) U.S. Cl. 702/20

(57) ABSTRACT

A causal network is used, which describes the regulatory genetic network of a cell such that nodes of the causal network represent genes of the regulatory genetic network and connectors of the causal network represent regulatory interactions between the genes of the regulatory genetic network. This causal network is adapted to the regulatory genetic network using a structure learning method. Using prior knowledge about a selected regulatory interaction between two genes, an a-priori information is determined for the connector representing the selected regulatory interaction. The a-priori information is taken into account for adapting the causal network to the regulatory genetic network using a structure learning method.

The present application hereby claims priority under 35 U.S.C. §119 on German patent application number DE 10 2004 030 296.0 filed Jun. 23, 2004, the entire contents of which is hereby incorporated herein by reference.

### FIELD

The invention generally relates to an analysis of a regulatory genetic network of a cell using a statistical method.

### BACKGROUND

Fundamentals of a regulatory genetic network of a cell are known from [1]. Such a regulatory genetic network should be taken in this document to include, in particular, regulatory interactions between genes of a cell.

A genome, i.e. the human genetic substance, is estimated to include 20,000 to 40,000 genes, of which a biologically specified number in each case—depending on a specialization of a cell—are present in the cell in the form of a DNA or a part of a DNA.

A not necessarily contiguous section of this DNA containing the genetic code for a protein or also for a group of proteins or for creating a protein or a group of proteins is designated as a gene here. Overall the genes contain a genetic code for around a million proteins.

An interplay or the interactions between the genes as well as with the proteins represents the most important part of a machinery (regulatory genetic network) which underlies the development of a human body from a fertilized minicell as well as all bodily functions.

It is also known from [1] that so-called gene expression rates which form a gene expression pattern supply a description or representation of a regulatory genetic network or of a current status of the regulatory genetic network.

In simple terms or expressed more clearly the gene expression pattern of a cell thus represents a state of the regulatory genetic network of this cell.

It is further known that by using high-throughput gene expression measurements (microarray data) these gene expression rates can be measured. The microarray data in its turn describes snapshots of the gene expression pattern.

It is further known that so called functional paths in a specific cell or of a specific tissue describe or reflect processes of a metabolism, of a gene regulation, of a transport and of a signal transduction.

Basically, cellular, molecular relationships can be subdivided into direct and indirect protein-protein interactions.

a) Indirect Protein-Protein Interactions (FIG. 1, FIG. 2)

a1) Metabolism and Metabolism Paths

The metabolism can be defined as the sum of the enzyme catalyzed actions occurring in a cell.

In this case the metabolism can be subdivided into conceptional units, known as metabolism paths, which are intermeshed with each other through common substrates in complex networks (cf. FIG. 4).

The metabolism has two main functions:
(1) The metabolism delivers energy required to maintain an internal composition of the cell and to support its functions.
(2) The metabolism also delivers metabolites which the cell needs to synthesize its components and products.

The set of all possible metabolism reactions can be represented as a graph (FIG. 4) with connectors and nodes. The metabolism graph here includes two types of nodes, namely, metabolites and enzymes. The connectors of the graphs represent substrate reactions and reaction-product relationships.

FIG. 1 shows—as a graphical illustration—an indirect protein-protein interaction in which an enzyme e2 1000 is regulated by an enzyme e1 1100 with the aid of a substrate s1 1200 in cross section.

Thus, according to FIG. 1, enzyme e1 1100 catalyzes the substrate s1 1200 which is needed by enzyme e2 1000 1102. 1101, 1102 e1 1100 thus interact with e2 1000 through substrate s1 1200. Since almost all enzymes are proteins, this can be seen as an indirect protein-protein interaction.

a2) Regulation of the Gene Expression

A basic question about to gene expression relates to the factors which control it. The gene expression is regulated on many molecular levels, beginning with the DNA level, through DNA winding processes, up to mRNA level by transcription regulation.

FIG. 2 shows—illustrated graphically—how the regulation of the gene expression can be interpreted as an indirect protein-protein interaction.

Thus 2101 in FIG. 2 a protein p1 2000 regulates the expression of a gene 2200, which in its turn encodes for a protein p2 2100 2102. Illustrated graphically it appears thus that p1 2000 interacts indirectly 210 with p2 2100.

b) Direct Protein-Protein Interaction (FIG. 3)

Many cell processes require specific interactions between different proteins.

What is known as a posttranslational modification serves as an important mechanism for modulation of the construction, the function, the activity and the half-life period of many proteins.

A phosphorylation, i.e. the covalent bonding of a phosphate group to either Serine, Threonine or Tyrosine, is the most frequent modification.

The phosphorylation, representable as a case of direct protein-protein interaction, is shown—illustrated graphically—in FIG. 3.

Thus in accordance with FIG. 3, a protein p2 3100 is modified 3201 by a protein p1 3000 by binding 3101 of a phosphate group p+3200 (phosphorylation). This interaction can be viewed as a direct protein-protein interaction.

Collections of genetic-biological information, such as a database TRANSFAC or a database Kyoto Encyclopedia of Genes and Genomes (KEGG), are known from literature.

TRANSFAC is a database of eukaryotic, cis-active regulatory DNA elements and transactive factors covering everything from yeast to human beings.

TRANSFAC provides information about transcription factors, their genome binding points and also their DNA binding profile.

The central part of the database consists of the description of specific protein interactions which are of regulatory significance for transcription.

The TRANSFAC data is generally taken from the original literature, also occasionally from other collections [15, 16] which contain suitable data.

The Kyoto Encyclopedia of Genes and Genomes (KEGG) is an attempt to computerize current knowledge of molecular and cell biology in relation to information paths including interacting molecules or genes and to provide links from the gene catalogs made available by the genome sequencing projects.

FIG. 4 shows—illustrated graphically—an extract, i.e. an information path from the KEGG, of what is known as a Methionine biosynthesis path in S. cerevisiae 4000.

FIG. 4 shows symbolized metabolites 4100 as nodes. Reactions, shown by FIG. 4 as connectors 4200, are identified by the EC number 4300 of the reaction-specific enzyme.

The enzymes shown in S. cerevisiae are shown shaded 4400 in FIG. 4.

Many illnesses and malfunctions of the body are attributable to disturbances in the regulatory genetic network which is reflected by greatly changed gene expression behavior (gene expression rates) or a changed gene expression pattern of a cell.

An understanding of the regulatory genetic network thus represents an important step on the path to a characterization of the understanding of genetic mechanisms, as well as consequent identification of what are known as dominant or malfunction-initiating genes underlying the illnesses or malfunctions.

In cancer research for example suppressing genes can play a key role in the identification of growths and tumors, the knowledge of new potential oncogenes and their interactions with other genes can be a contribution to discovering the basic principles (of cancers) which determine how normal cells change into malignant cancer cells.

Furthermore a quantitative understanding of the regulatory genetic network of a cell is necessary for developing improved medicaments and therapies for fighting genetic diseases.

Thus a number of medicaments act as agonists or antagonists of specific target proteins, i.e. they strengthen or weaken the function of a protein with corresponding effect on the regulatory genetic network with the aim of bringing this back into a normal function mode.

A description of a regulatory genetic network of a cell using a statistical method, a causal network is known from [2].

A causal network, a Bayesian network, is known from [3, 5].

Bayesian Network

A Bayesian network B is a specific type of presentation of a common multivariate probability density function of a set of variables X by a graphical model which consists of two parts.

It is defined by a directed acyclic graph, DAG) G—of the first component, in which each node i=1, . . . , n corresponds to a random variable Xᵢ.

The connectors between the nodes represent statistical dependencies and can be interpreted as causal relationships between them.

The second component of the Bayesian network is the set of conditional WDFs P(Xᵢ|Paᵢ, θ, G), which are parameterized by way of a vector θ.

These conditional WDFs specify the type of dependencies of the individual variables i of the set of its parents Paᵢ. Thus the common WDF can be broken down into the product form

$$P ( {X}_{1} , {X}_{2} , … , {X}_{n} ) = {\prod }_{i = 1}^{n} P ( {X}_{i} \vert P {a}_{i} , θ , G ) \\ ( Markov independency ) . \qquad ( 1 )$$

The DAG of a Bayesian network uniquely describes the conditional dependency and independency relationships between a set of variables, but by contrast a given statistical structure of the WDF does not result in any unique DAG.

Instead it can be shown that two DAGs describe one and the same WDF, if and only if they feature the same set of connectors and the same set of “colliders”, with a collider being a constellation in which at least two directed connectors lead to the same node.

Further information on the use of statistical methods, especially in the field of biologic and genetic knowledge is known from [11] to [14] and in particular represent an expert's specialist knowledge to be included.

### SUMMARY

An object of an embodiment of the invention is to specify a method which allows an analysis of a regulatory genetic network of a cell, for example represented by at least one gene expression pattern of the cell.

A further underlying object of an embodiment of the invention is to specify a method which makes it possible or which creates an instrument with which basic interrelationships of genetic, biological processes in a cell are analyzed and can be illustrated.

In addition, an embodiment of the invention is intended to make it possible to identify a specific gene such as a defective gene, for example an oncogene or tumor gene, in the regulatory genetic network of a cell.

Further an embodiment of the invention is designed to allow a simulation and/or an analysis of an effect of a medicament on the regulatory genetic network of a cell.

An object may be achieved by the method, the computer program product with program code segments and/or the computer program product for analysis of a regulatory genetic network of a cell.

With the underlying method of at least one embodiment for analysis of a regulatory genetic network of a cell, a causal network is used which describes the regulatory genetic network of the cell such that nodes of the causal network represent genes of the regulatory genetic network and connectors of the causal network represent regulatory interactions between the genes of the regulatory genetic network.

Using a structure learning process adapts this causal network to the regulatory genetic network, with the nodes and the connectors of the causal networks being adapted at least structurally to the regulatory genetic network of the cell.

Using prior knowledge about a selected regulatory interaction between two genes an a-priori information is determined for the connector representing the selected regulatory interaction.

This connector, i.e. the one representing the a-priori information for the selected regulatory interaction may now be taken into account for adapting the causal network to the regulatory genetic network using the structure learning method.

The computer program product with program code segments is set up to execute all the steps in accordance with the inventive method of at least one embodiment, when the program is executed on a computer.

The computer program product with program code segments stored in machine-readable form on a data medium is set up to execute all the steps in accordance with the method in accordance with at least one embodiment of the invention when the program is run on a computer.

The computer program product with program code segments set up to execute all steps in accordance with the inventive method of at least one embodiment when the program is run on a computer, as well as the computer program product with program code segments stored on a machine-readable medium, set up to execute all steps in accordance with the inventive method of at least one embodiment when the program is executed on a computer are especially suited to execute the method in accordance with at least one embodiment of the invention or of one of its further developments listed below.

The invention is based on non-trivial knowledge, its application and/or implementation.

It is thus recognized that probabilistic semantics of a causal network, such as a Bayesian network, is very well suited to analysis of gene expression rates, for example given in the form of microarray data, since it is adapted to the stochastic nature both of biological processes and also of experiments adversely affected by noise.

Furthermore, viewed in illustrative terms, an effect of an expression state of specific genes on a global gene expression pattern (inverse modeling) is estimated, in that a resulting gene expression pattern—obtainable from the causal network—is analyzed.

Furthermore the method for analysis of a regulatory genetic network of a cell is based on the non-trivial and inventive knowledge that by introducing a structure prior in a Bayesian estimator prior knowledge about regulatory relationships of the regulatory genetic network can be taken into account or incorporated.

Thus at least one embodiment of the invention can also be seen as illustrating estimation of regulatory relationships between the genes of an organism from statistical data, such as the gene expression data, while including imprecise prior knowledge about regulatory relationships.

The non-trivial introduction of a structure prior in a Bayesian estimator guides a data-driven estimation procedure by prior knowledge. The introduction of the prior covering the regulatory relationships between genes allows a degree of knowledge about the presence and the type of relationships to be defined.

The developments described below relate to both the method and to the arrangement.

Embodiments of the invention and the developments described below can be implemented both in software and also in hardware, for example by using a specific electrical circuit.

Further the realization of embodiments of the invention or of a development described below is possible through a computer-readable storage medium on which a computer program product with program code segments is stored which executes at least one embodiment of the invention or development.

Also at least one embodiment of the invention or any development of it described below can be realized by a computer program product which features a storage medium on which a computer program product with program code segments is stored which executes at least one embodiment of the invention or development.

In a preferred development the prior knowledge is information about a functional path, especially a metabolism path, of a cell. Such a functional path is especially well suited for describing the interactions between metabolism products.

The functional path can also describe an interaction between a gene regulation, a transport or a signal transduction.

Preferably the a-priori information is at least one a-priori probability of the presence or absence of a Markov relationship between nodes of the causal network or for the presence or absence of a Markov relationship between connectors of the causal network

In the preferred development a number of items of a-priori information are determined for a number of connectors representing the relevant selected regulatory interactions.

Also during the determination of the a-priori information using the prior knowledge the regulatory interactions can be interpreted as at least part of a directed graph, with the part of the directed graph able to be a direct or indirect directed protein-protein interaction.

The structure learning can be undertaken using an evaluation function, for example a Bayesian score, which is especially formed from a marginal likelihood and an a-priori likelihood of a structures of the causal network.

In this case an assumption can be useful, namely that the a-priori likelihood of the structure of the causal network can be broken down.

In a further development a Bayesian network is used as the causal network of which the structure is in particular a DAG (directed acyclic graph) type.

There can also be provision for the causal network to be trained using gene expression patterns, with the nodes and the connectors of the causal networks being adapted.

It is further worthwhile for the gene expression patterns to be determined using a DNA microarray technique.

In one embodiment the gene expression patterns for the training are gene expression patterns of a genetic regulatory network of a diseased cell.

Here for example the diseased call can be a cancer cell, especially a an oncocell with ALL (Acute Lymphoblastic Leukemia).

Furthermore the diseased cell can feature an oncogene, especially an ALL oncogene.

Furthermore the inventive procedure or development of at least one embodiment is particularly suitable for identifying a dominant gene and/or a degenerated/mutated/diseased oncogenic/tumor suppressor gene.

It is also suitable for identifying a tumor cell, for example in connection with cancer detection.

Further at least one embodiment of the inventive method is especially suited to analyzing the causes of an abnormal gene expression pattern/gene expression rate.

It can also be used for a simulation and/or analysis of the effects of a medicament.

### BRIEF DESCRIPTION OF THE DRAWINGS

Further advantages, features and possible applications of the present invention are produced from the description of the example embodiments below which refer to the Figures.

The Figures show

FIG. 1 a sketch showing a direct protein-protein interaction in which an enzyme e2 is regulated by an enzyme e1 with the aid of a substrate;

FIG. 2 sketch showing a direct protein-protein interaction in which a protein p1 interacts with a protein p2 through regulation of the gene expression;

FIG. 3 sketch showing a direct protein-protein interaction in which a protein p1 interacts with a protein p3 through phosphorylation;

FIG. 4 a sketch which shows an extract, an information path from the KEGG, of a what is known as a Methionine biosynthesis path in S. cerevisiae;

FIG. 5 a sketch which shows the metabolism path from a sulfur metabolism of S. cerevisiae.

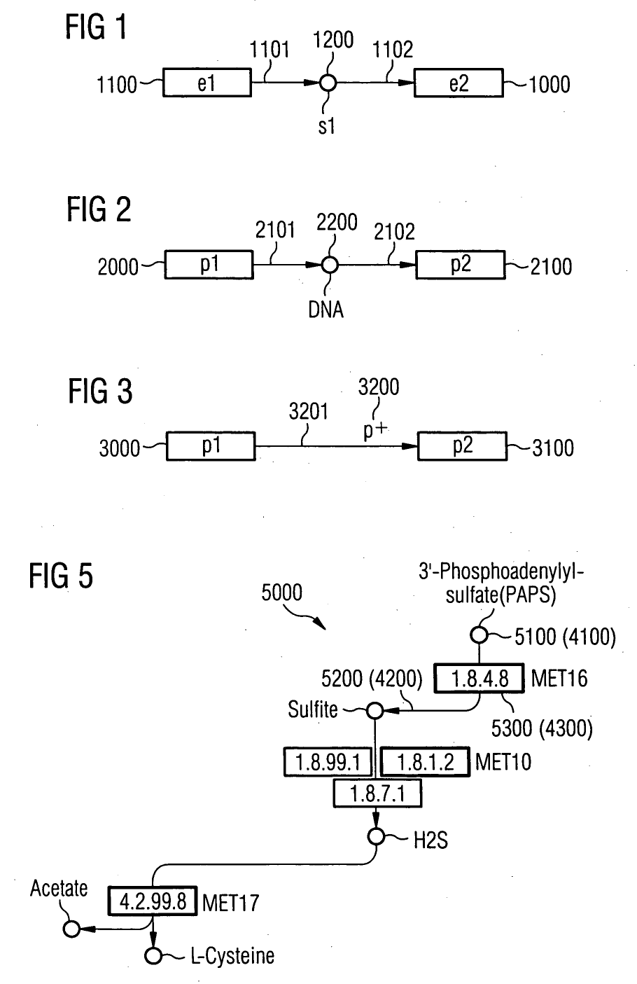

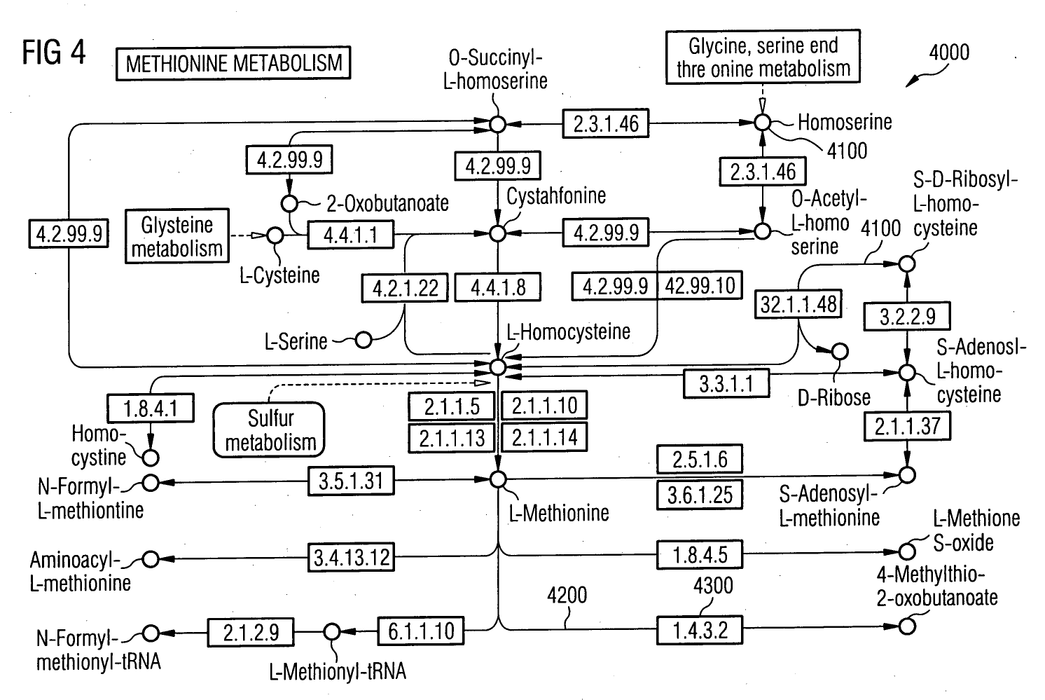

### DETAILED DESCRIPTION OF THE EXAMPLE EMBODIMENTS

Example embodiment: Analysis of a regulatory genetic network using causal networks—Integration of biologic a-priori information for learning control networks

Introduction/Overview

Cellular molecular network systems arise through complex interactions between proteins, DNA, RNA and other molecules.

The complex regulatory network between genes and proteins, the genetic network forms a central part of this cellular life mechanism, with its different operating modes controlling the plurality of biochemical processes in a living cell.

A main interest of the post-genome area is thus to understand the structures and function of genetic networks in normal cell operation, for pathological states after gene damage and in the response to attacks from outside, such as treatment with medicines or extracellular signals.

In the embodied procedure Bayesian statistics are applied to analysis and mapping the topology of genetic regulatory networks.

By using learning Bayesian networks [3, 6, 7, 9] the structures of a genetic network are estimated from a set of gene expression measurements [4], with the Bayesian network mapping the genetic network structures and/or functions.

Further, with an embodiment of the inventive method described here the network topology, for example of micro array data, is learned by way of Bayesian statistics—and thereby the regulatory genetic network (functions) emulated or created—, with a particular capability of Bayesian statistics being used and included here.

Bayesian statistics allow prior knowledge or a-priori knowledge to be integrated into the adaptation of a Bayesian network to the topology of a genetic regulatory network, for example known interactions between specific genes.

The integration of a-priori knowledge can then help to punish structures which do not make any biological sense and by contrast give preference to structures which are biologically more sensible.

Methods

Bayesian Network from Expression Patterns

Bayesian Network

A density estimation of gene expression data is described in [7, 8, 9] and is only briefly summarized here.

A Bayesian network B involves a specific, two-part form of representation of a shared multivariate probability density function, pdf P of a set of variables X by way of a graphical model.

It is defined by a directed acyclic graph, DAG) G of the first component, in which each node i=1, . . . , n corresponds to a random variable Xᵢ. The connectors between the nodes represent statistical dependencies and can be interpreted under specific conditions [10] as causal relationships between them.

The set of parents Pa(i) of i is determined by the graph structure G as nodes which send out a directed connector to i.

The second part of the Bayesian network includes a set of conditional pdfs P(Xᵢ|Paᵢ, θ, G), which are parameterized by a vector θ. The connection between G and θ is defined by a Markov independency. Each variable Xᵢ is, for given parent nodes Paᵢ in G, independent of its non-succession.

These conditional pdfs determine the types of dependency for each variable i on its parents Paᵢ. Thus the shared pdf can be broken down into the product form

$$P ( {X}_{1} , {X}_{2} , … , {X}_{n} ) = {\prod }_{i = 1}^{n} P ( {X}_{i} \vert P {a}_{i} , θ , G ) \qquad ( 1 )$$

The DAG of a Bayesian network uniquely describes the conditional dependency and independency relationships between a set of variables, by contrast a given statistical structure of the pdf cannot be used to conclude a unique DAG.

Instead it can be shown that two DAGs describe the same pdf if and only if they feature the same set of connectors and the same set of colliders, with a collider being a constellation for which at least two directed connectors converge in same node.

DAGs of the same equivalence class can be represented by a single partial directed graph, PDAG) with all reversible connectors being drawn in undirected form.

In the modeling of a regulatory genetic network by a Bayesian network the genes or their corresponding proteins are symbolized by nodes. It is assumed here that the regulatory mechanisms are reflected by connectors between two nodes.

If the connectors are directed, this is interpreted as the direction of the regulation. The quality of the regulation (simplification or suppression) is encoded in the conditional probability distribution of the genes involved by specifying its regulators.

Structural Learning

The learning of Bayesian networks from data is has become an increasingly active area of research and can be subdivided into two problem definitions.

In the first case is the network structure is already known and only the parameters have to be learned from the data set.

The second task, structural learning, is more difficult, since as well as the parameter values the network structure has to be learned from the data set (structural learning).

The method of structural learning can be specified as follows: Let it be assumed that D={d¹, d², . . . , d^{N}} is a metadata set including N independent observations, with for each data point involving an n-dimensional vector with the components d^{l}={d^{l}₁, . . . , d^{l}ₙ}, l=1, . . . , N.

Evaluation Function (Bayesian Score)

To assess the quality of adaptation of a network in relation to the data set D the graph G is assigned a value S(G) (Bayesian score) by a statistically motivated evaluation function S.

This evaluation function S is derived from the methods of the Bayesian statistics. It is proportional to the a-posteriori likelihood of a network structure for given data:

$$S ( G \vert D ) = \frac{P ( D \vert G ) P ( G )}{P ( D )} \qquad ( 2 )$$

P(D|G) is the marginal likelihood, P(G) the a-priori likelihood of the structure and P(D) is called evidence.

Since the evidence P(D) is constant across the different structures, it can be ignored.

Furthermore the a-priori likelihood of the structure P(G) for non-available a-priori knowledge about the structures is replaced by an non-informative a-priori likelihood, that is P(G)=const.

If both a-priori-likelihoods are ignored, the problem is now reduced to finding the structure with the best marginal likelihood for the corresponding data.

In other words: how likely is it that the data has been generated from the structure.
P(D|G)=∫P(D|Θ, G)P(Σ|G)dΘ (3)

For the given equation 1 P(D|Θ, G) can be transposed:

$$P ( D \vert Θ , G ) = {\prod }_{l = 1}^{N} {\prod }_{i = 1}^{n} P ( {d}_{i}^{l} \vert P {a}_{i} ( {d}^{l} ) , G , Θ ) \qquad ( 4 )$$

For a given multinomial model of n variables, as is known from literature, a series of assumptions can be made, these being complete data, parameter independence and modularity of the parameters.

Thus and in combination with a-priori Dirichlet distributions equation 4 can be transposed:

$$P ( D \vert G ) = {\prod }_{i = 1}^{n} {\prod }_{j = 1}^{{q}_{i}} \frac{Γ ( {N}_{ij}^{′} )}{Γ ( {N}_{ij}^{′} + {N}_{ij} )} {\prod }_{k = 1}^{{r}_{i}} \frac{Γ ( {N}_{ijk}^{′} + {N}_{ijk} )}{Γ ( {N}_{ijk}^{′} )} \qquad ( 5 )$$

with rᵢ designating the set of values that the variable Xᵢ can assume, and qᵢ designating a set of values that the filter of Xᵢ can assume.

$$Γ ( x ) = {\int }_{0}^{∞} {t}^{x - 1} {ⅇ}^{- t} ⅆ t$$

is the Gamma function; for positive integers Γ(χ)=(χ−1)!.

Nᵢⱼₖ designates the number of cases in the data set D, for which d^{l}ᵢ,=k and Paᵢ(d^{l})=j, and

$${N}_{ij} = {\sum }_{k = 1}^{{r}_{i}} {N}_{ijk} .$$

N′ᵢⱼₖ expresses parameters of the a-priori-Dirichlet distributions and

$${N}_{ij}^{′} = {\sum }_{k} {N}_{ijk}^{′} . {N}_{ijk} = \frac{1}{{q}_{i} {r}_{i}} ,$$

which are often used a-priori as non-informative parameters.
A-Priori Likelihood of the Structure

One advantage of Bayesian statistics lies in the capability of combining a-priori knowledge with information obtained from the data.

Thus in areas in which a-priori knowledge about the structures is available, this a-priori knowledge is integrated via the a-priori likelihood of the structure P(G) into the structure learning in accordance with the evaluation function for structure learning according to equation 2 or equation 6.

Especially when the likelihood distribution of the data set is very sparse, such as for example in micro array trials, the inclusion of a-priori knowledge in the structure learning algorithm can considerably enhance its power.

In this case the evaluation function S divides into two parts:
S(G|D)=P(D|G)P(G) (6),
with P(D|G), as described above and able to be calculated according to equation 5, being the marginal likelihood and P(G) the a-priori likelihood of the structure.

For simplicity's sake it is assumed that the a-priori likelihood of the structure can be broken down. Then each connector from node i to node j can be provided with a likelihood Pⱼᵢ.

This is the common likelihood p(i→j, [symbol] j→i), with p(i→j, j→i)=0— because of the conditions of acyclicity of the graph.

Thus the a-priori likelihood of the structure between node i and node j can be described with three expressions: pⱼᵢ, pᵢⱼ and 1−(pⱼᵢ+pᵢⱼ), the a-priori-likelihood for the nonpresence of a Markov relationship between node i and node j.

If a-priori no information is available about the Markov relationship between node i and node j all three expressions described above have the same likelihood of 1/3.

If it is known from prior knowledge that there must be a connector between i and j but no information about the direction of the connector is available, pⱼᵢ and pᵢⱼ have the same value of 1/2.

Otherwise, i.e. if the direction information for the connectors i,j, is present, the relevant pⱼᵢ or pᵢⱼ has the value of 1.

Structure Example

The matrix P(G) represents the a-priori information about the structure G of a Bayesian network B which includes 3 variables X₁, X₂ and X₃.

$$P ( G ) = ( \begin{matrix}0 & \frac{1}{3} & \frac{1}{2} \\ \frac{1}{3} & 0 & 1 \\ \frac{1}{2} & 0 & 0\end{matrix} )$$

For two Markov relationships, namely X₂-X₃ and X₁-X₃, there is a-priori-information available.

For X₁-X₃ the a-prior information, indicates that a Markov relationship must exist between them and must exist from X₂ to X₃, the same applies for X₁-X₃, but without knowledge about the direction.

In this case, from the 25 possible DAG 5 reach the maximum a-priori-likelihood of 1*1/3*1/2=0.16.

These graphs have the following structure characteristics: X₂→X₃, X₁→X₃ or X₃→X₁ and any given relationship between X₁ and X₂.

Now together with the marginal likelihood P(D|G), as described above and able to be computed according to equation 5, the evaluation function S can be determined for structure learning in accordance with equation 6, in order to—during structure learning—identify the network structure which best maps the data.

A-Priori Structure Likelihood from Biology

All relationships of genetic biological information, such as the database TRANFAC or the database Kyoto Encyclopedia of Genes and Genomes (KEGG), have already been given above which provide a vast amount of biological data which can be used as structural a-priori knowledge.

It will be shown below how prior knowledge or a-priori knowledge from molecular biology can be integrated into a structure learning algorithm.

FIG. 5 shows a metabolism path 5000 from the sulfur metabolism of S. cerevisiae.

The path 5000 can be interpreted as a chain of indirect protein-protein interactions with each metabolite 5100 being the product of an enzymatic reaction 5200 as well as the substrate for the following enzyme 5300.

Since enzymes can only catalyze their reaction in one direction, the path 5000 can be represented as a directed graph which can be used as structural a-priori knowledge.

The corresponding graph G consists of 3 variables x={MET16, MET10, MET17}. The a-priori likelihood P(G) of the structure can be transposed taking account of the prior knowledge from the sources given above:

$$P ( G ) = ( \begin{matrix}0 & 0.8 & 0.3 \\ 0.1 & 0 & 0.8 \\ 0.3 & 0.1 & 0\end{matrix} )$$

In accordance with the biological information from sources given above p_{MET16 MET10} and P_{MET10 MET17} (rounded) with 0.8 a high a-priori likelihood can be assumed.

The corresponding reversed connectors have a low of a-priori likelihood of 0.1 (rounded) since, as already explained above, enzymes are only active in one direction.

Only for the Markov relationship between MET16 and MET17, can no a-priori information be taken from the above sources so that the likelihood for all three likelihoods (pⱼᵢ, pᵢⱼ, 1−(pⱼᵢ+pᵢⱼ)) amounts to rounded 0.3.

Furthermore, now in accordance with equation 2 or equation 6, together with the marginal likelihood in accordance with equation 5, structured learning can be evaluated to identify the network structure which best maps the data.

Finally a particular advantage of Bayesian statistics will be mentioned separately: Bayesian statistics allows a-priori knowledge to be combined with information obtained from data.

Thus in areas in which a-priori knowledge about the structures is available, this a-priori knowledge is integrated via the a-priori likelihood of the structure P(G) into the structure learning in accordance with the evaluation function for structure learning according to equation 2 or equation 6.

The integration of a-priori knowledge can then help to punish structures which do not make any biological sense, e.g. connector MET10→shown here? MET16, and by contrast to give preference to biologically sensible connectors, for example MET16→MET10.

### SUMMARY

The researching and the understanding of networks of molecular interactions, their modes of operation under different circumstances and their response to external signals is the main requirement of the post-genome era.

The data pool for reconstructing such networks is growing rapidly as a result of techniques with high throughput. The networks obtained are mostly very complex so that the relevant information about the mapped system and its components is not intuitively visible and makes additional extensive statistical analysis necessary.

In a procedure described in accordance with the embodiment a network topology, for example including microarray data, is learned by way of Bayesian statistics and the regulatory genetic network is thus (functionally) mapped or created. In this case there is recourse to a particular capability of Bayesian statistics or for structure learning a prior knowledge or a-priori knowledge is incorporated.

The integration of a-priori knowledge can then help to punish structures which do not make any biological sense, and by contrast to give preference to biologically sensible structures.

The following publications are cited in this document:
[1] Stetter Martin et al., Large-Scale Computational Modeling of Genetic regulatory Networks, Kluwer Academic Publisher, Netherlands, 2004 Edition
[2] Publication number DE 10159262.0
[3] F. W. Jensen, F. V. (1996), An introduction to Bayesian networks, UCL Press, London; 178 pages
[4] E.-J. Yeoh, M. E. Ross, S. A. Shurtleff, W. K. Williams, D. Petal et al. (2002), Classification, subtype discovery, and prediction of outcome in pediatric acute lymphoblastic leukemia by gene compression profile. Cancer cell 1:133-143
[5] D. Heckerman, D. Geiger and D. Chickering (1995), Learning Bayesian networks: The combination of knowledge and statistical data, Machine Learning 20:197-243
[6] Friedman, N., Goldszmidt, M. and Wyner, A. (1999). Data analysis with Bayesian networks: a bootstrap approach, pp. 196-205
[7] Friedman, N., Linial, M., Nachman, I. and Pe'er, D. (2000). Using Bayesian networks to analyze compression data., J. Comput. Biology 7:601-620
[8] Dejori, M. and Stetter, M. (2003). Bayesian inference of genetic networks from gene-compression data: convergence and reliability, Proceedings of the 2003 International Conference on Artificial Intelligence (IC-A1 '03), pp. 323-327
[9] Heckerman, D., Geiger, D. and Chickering, D. (1995). Learning Bayesian networks: The combination of knowledge and statistical data, Machine Learning 20:197-243
[10] Lauritzen, S. L. (1999). Causal interference from graphical model, Technical report pp. R-99-2021
[11] Gavin, A. C., Bosche, M., Krause, R. and Grandi, P. (2002). Functional organization of the yeast proteome by systematic analysis of protein complexes, Nature 415:378-381
[12] Baldi, P. and Hatfield, G. W. (2002). DNA microarrays and gene compression, Cambridge University Press, Cambridge Mass.
[13] Stetter, M., Deco, G. and Dejori, M. (2003). Large-scale computational modeling of genetic regulatory networks, AI Review, 20: 75-93
[14] van Duk, M. A., Voorhoeve, P. M. and Murre, C. (1993). PBXL is converted into a transcriptional activator upon α-quiring the N-terminal region of E2A in pre-b-cell c, Proc. Natl. Acad. Sci. USA 90: 6061-6065
[15] Faisst and Meyer, Nucleic Acids Res. 20:3-26, 1992
[16] Dhawale and Lande, Nucleic Acids Res. 21:5537-5546, 1994).

Further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, such as floppy disks (trademark), cassette tapes, and removable hard disks; media with a built-in rewriteable non-volatile memory, such as memory cards; and media with a built-in ROM, such as ROM cassettes.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. Method for adapting a causal network to a regulatory genetic network of a cell, the causal network describing the regulatory genetic network of the cell such that nodes of the causal network represent genes of the regulatory genetic network and connectors of the causal network represent regulatory interactions between the genes of the regulatory genetic network, the method comprising:
a) determining, using prior knowledge of a selected regulatory interaction between two genes, an a-priori information for the connectors representing selected regulatory interactions;
b) adapting the node and the connectors of the causal network at least structurally to the regulatory genetic network of the cell using a structure learning process, taking the determined a-priori information into account.

2. Method according to claim 1, wherein the prior knowledge is information about the functional path.

3. Method according to claim 1, wherein the functional path describes an interaction between metabolism products, of a gene regulation, of at least one of a transport channel and a signal transduction.

4. Method according to claim 1, wherein the a-priori information is at least an a-priori likelihood of the presence of a Markov relationships between at least one of nodes of the causal networks and a connector of the causal network.

5. Method according to claim 1, wherein a number of items of a-priori information for a number of connectors representing the selected regulatory interactions is determined.

6. Method according to claim 1, wherein, for the determination of a-priori information using the prior knowledge, the regulatory interaction is interpreted as at least part of a directed graph.

7. Method in accordance with claim 6, wherein the part of the directed graph is a directed protein-protein interaction.

8. Method in accordance with claim 1, wherein a Bayesian network is used as a causal network.

9. Method according to claim 1, wherein the structure learning is executed using an evaluation function.

10. Method according to claim 1, wherein the a-priori likelihood of the structure of the causal network can be broken down.

11. Method according to claim 1, wherein the causal network must be trained using gene expression patterns, with the node and the connectors of the causal network being adapted.

12. Method in accordance with claim 11, wherein the gene expression patterns are determined using a DNA microarray technique.

13. Method in accordance with claim 11, wherein the gene expression patterns for the training gene expression pattern are a genetic regulatory network of a diseased cell.

14. Method in accordance with claim 13, wherein the diseased cell is an oncocell especially an oncocell with ALL (acute lymphoblastic leukemia) which in particular features an oncogene, especially an ALL oncogene.

15. Method of identifying a dominant gene, using the method according to claim 1.

16. Method of identifying at least one of a degenerated/mutated/diseased/oncogenic/tumor-suppressor cell and/or gene, using the method according to claim 1.

17. Method of identifying a tumor cell, using the method according to claim 1.

18. Method of detecting cancer, using the method according to claim 1.

19. Method of at least one of simulating and analyzing an effect of a medicament, using the method according to claim 1.

20. Computer program product with program code segments, to execute the method according to claim 1 when the program is executed on a computer.

21. Machine-readable data medium with program code segments, to execute the method according to claim 1 when the program is executed on a computer.

22. Computer program product with program segments stored on a machine-readable data medium, to perform the method according to claim 1, when the program is executed on a computer.

23. Method according to claim 1, wherein the prior knowledge is information about a metabolism path of a cell.

24. Method in accordance with claim 1, wherein a Bayesian network is used as a causal network, of which the structure is of a type DAG (directed acyclic graph).

25. Method in accordance with claim 13, wherein the diseased cell is an oncocell with ALL (acute lymphoblastic leukemia), which features an ALL oncogene.

26. Method of claim 1, further comprising:
analyzing the regulatory genetic network of the cell using the adapted causal network.
